(19) United States
(12) Patent Application Publication
(10) Pub. No.: US 2006/0199234 A1
(43) Pub. Date: Sep. 7, 2006

(54) Method of diagnosing wood decay and decay diagnostic agent

(76) Inventors: Keijitsu Tanaka, Ibaraki (JP); Takashi Echigo, Ibaraki (JP); Masahiko Hiramoto, Ibaraki (JP)

Correspondence Address: SUGHRUE MION, PLLC, 2100 PENNSYLVANIA AVENUE, N.W., SUITE 800, WASHINGTON, DC, 20037, US

(21) Appl. No.: 10/565,554

(22) PCT Filed: Jun. 3, 2004
(86) PCT No.: PCT/JP04/08052

§ 371 Date: Jan. 23, 2006

(30) Foreign Application Priority Data
Jul. 25, 2003 (JP) 2003-201951

(51) Int. Cl. G01N 33/569; C07K 16/14

(52) U.S. Cl. 435/7.31; 530/388.5

(57) ABSTRACT

The invention provides a simple method which enables precise diagnosis of wood decay at an early stage of decay and an agent used for the diagnosis method. The invention provides a method for diagnosing wood decay, wherein wood decay is judged by contacting extract of wood to be examined (test subject wood) with an antibody obtained by sensitizing an animal to an antigen which is a protein having a molecular weight of 1,000 to 100,000 obtained by culturing a naturally occurring wood-destroying fungus and an agent for the diagnosis method.

### TECHNICAL FILED

The present invention relates to a method for diagnosing decay in wood and an agent for diagnosing wood decay used in the method. Further, the present invention relates to a simple method for diagnosing decay in wood and a test kit used in the method for diagnosing decay in wood.

### BACKGROUND ART

Wood is an excellent material and used in a wide range of fields, mainly as a building material, however wood has a disadvantage that it is susceptible to the breakdown of materials by microbial action (biodeterioration).

Biodeterioration of wood is roughly classified into feeding damage by insects such as termites and decay caused by microorganisms such as wood destroying fungi. The former damage can be judged by visual observation and/or simple test. In most cases of the latter damage, where the damage is caused by growth of wood-destroying fungi inside the wood material, the damage cannot be judged by visual observation only. Moreover, since growth of wood-destroying fungi is accompanied by metabolic decomposition of wood constituents such as cellulose, hemicellulose and lignin, a decay degree of the wood, which is generally evaluated as weight loss rate, becomes several to 10% or so and strength of the wood decreases by half. In particular, since wood decay caused brown-rot fungi is frequent in building materials, there is a demand for a method of precisely detecting wood decay at an early stage of decay.

In order to solve this problem, the material needs to be tested as to presence or absence of decay at regular intervals. In conventional detecting methods, (1) examination by percussion or touch, (2) culturing fungi taken out from a sample of decayed wood, (3) determination according to resistance observed in driving a nail-like tool (PILODYN) into the material, (4) determination according to change in ultrasonic transmission speed or with acoustic emission (AE) and the like, have been proposed.

However, with respect to method (1), wood decay at an early stage cannot be detected by examination by percussion or touch, and determination by (1) is ambiguous. With respect to method (2), it takes long to obtain results of culturing fungi and such a method requires expert technique. With respect to method (3), although a PILODYN, which drives a steel pin by a spring into wood and measures the degree of decay from the penetration depth, the determination depends on density and quality of the sample wood and therefore, quantitative results cannot be obtained and decay cannot be detected at an early stage. With respect to method (4), decay inspection by sonic wave or by acoustic emission (AE) not only uses a considerably large apparatus and is expensive, but also requires expert knowledge and technique. Thus, all the conventional decay-detection approaches have disadvantages and none of them is widely used.

Another detection approach using antigen-antibody reaction has been proposed.

For example, in J. W. Palfreyman et al., “Immunological Methods for the Detection and Characterization of Wood Basidiomycetes”, Biodeterior 7, 709-713 (1988), it is described that fungi can be detected by antiserum prepared by using Lentinus lepideus or Coriolus versicolor (Trametes versicolor). According to this document, the antiserum can detect other Basidiomycetes or some other mold fungi. However, the document does not describe names of the fungi responding to the antiserum. Moreover, the document describes that various fungi responded to control antiserum (antiserum not inoculated with fungi) Generally, many kinds of fungi including mold fungi are present on the surface of a wood material suspected of being decayed and therefore, the method involving responses from those fungi present on the surface is not effective as a method for detecting wood-destroying fungi.

In C. A. Clausen et al., “Early detection of brown-rot decay in southern yellow pine using immunodiagnostic procedures”, Wood Sci. Technol. 26, 1-8 (1991), it is described that an antiserum prepared by mixing multiple kinds of antigens derived from wood-destroying fungi together and inoculating a rabbit with it can detect those multiple kinds of wood-destroying fungi. However, this method, which requires culturing multiple kinds of fungi, is cumbersome. Moreover, in this document, comparison is made between weight loss, enzyme-linked immunosorbent assay (ELISA) method, aggregation method (activated carbon/latex) and dot blotting method. In aggregation method, although reaction can be observed even when the decay degree is low, the obtained result can be negative even when the decay degree is high. Accordingly, the method possesses low reliability. In ELISA method, some kinds of wood-destroying fungi cannot be detected while in dot blotting method, detection sensitivity is low as a whole.

In addition, in Yoon Soo Kim et al., “The Use of ELISA for the Selection of White- and Brown-Rot Fungi”, Holzforschung 45, 403-406 (1991), ELISA method using an antiserum obtained from a rabbit sensitized with wood-destroying fungi is studied, and it is described that against an antiserum obtained by using a specific wood-destroying fungus, the same wood-destroying fungus only is highly responsive. As an exception, the document describes that against an antiserum based on Postia placenta, other kinds of fungi are responsive, however the mode of the response varies depending on implementing conditions of ELISA method. Postia placenta is a wood-destroying fungus which does not inhabit in Japan and since there is a control measure to prevent the fungus from entering in the country, the fungus is not available in Japan.

U.S. Pat. No. 5,563,040 proposes a more reliable detection method, in consideration for conventional methods lacking in reliability and nonspecific nature. Specifically, the patent discloses a method where a capture zone and a carrier zone are provided in a polyester cloth, the first antibody (monoclonal antibody) which is specific for a wood-destroying fungus antigen is fixed in the capture zone while the second antibody (polyclonal antibody) against the wood-destroying fungus antigen is fixed on colored beads in the carrier zone. In test procedures, the polyester cloth is soaked in an extract liquid of sample wood with the carrier zone being kept down. When a wood-destroying fungus antigen is present in the extract, the antigen is bound to the second antibody on the colored beads and the antigen in the liquid brings the second antibody upward together with the colored beads in accordance with the liquid being soaked up by capillary action, and then the antigen is further bound to the first antibody in the carrier zone to form the first antibody/the antigen/the second antibody (colored beads) complex. By observing a line thus formed by the colored beads captured by the capture zone, presence or absence of wood-destroying fungi is determined. However, in this document, only the effect obtained in a case where an antibody against xylanase derived from Postia placenta is used as a monoclonal antibody is confirmed. However, the document does not include description about Fomitopsis palustris, Trametes versicolor and Serpula lachrymans, which are representative types of wood-destroying fungi in Europe, USA and Japan and therefore, the method cannot be said to be useful in detection of overall kinds of typical wood-destroying fungi.

Thus, in conventional detection methods using antigen-antibody reaction, the object to detect various kinds of wood-destroying fungi simultaneously without detecting fungi other than wood-destroying fungi cannot be attained.

### DISCLOSURE OF THE INVENTION

Accordingly, an object of the present invention is to provide a diagnosis method for detecting wood decay caused by various kinds of wood-destroying fungi simultaneously without detecting fungi other than wood-destroying fungi, and also provide an agent used in the diagnosis method.

The present inventors have obtained the results that an antibody obtained by sensitizing an animal with an antigen which is a protein having a molecular weight of 1,000 to 100,000 obtained by culturing a naturally occurring wood-destroying fungus such as Fomitopsis palustris is widely reactive not only with extract of wood decayed by the wood-destroying fungi used to obtain the protein but also extract of wood decayed by other wood-destroying fungi while the antibody shows no significant reactivity with extract of wood where fungi other than wood-destroying fungi are growing, and thus completed the invention.

That is, the present invention provides a method for diagnosing wood decay, an agent used in the method of wood-decay diagnosis, a simple a method for diagnosing decay in wood and a test kit used in the method for diagnosing decay in wood.

1. A method for diagnosing wood decay, wherein wood decay is judged through antigen-antibody reaction of contacting extract of wood to be examined with an antibody obtained by sensitizing an animal with an antigen which is a protein having a molecular weight of 1,000 to 100,000 obtained by culturing a wood-destroying fungus.

2. The method for diagnosing wood decay according to 1, wherein decay by multiple kinds of wood-destroying fungi is judged through antigen-antibody reaction of contacting extract of wood to be examined with an antibody obtained by sensitizing an animal with an antigen which is a protein having a molecular weight of 1,000 to 100,000 obtained by liquid-culturing a wood-destroying fungus.

3. The method for diagnosing wood decay according to 1 or 2, wherein the protein is obtained by culturing one wood-destroying fungus selected from the group consisting of Fomitopsis palustris, Gloeophyllum trabeum, Coniophora puteana, Serpula lacrymans, Trametes versicolor and Gloeophyllum sepiarium.

4. The method for diagnosing wood decay according to 3, wherein the protein is obtained by culturing Fomitopsis palustris.

5. The method for diagnosing wood decay according to 1 or 2, detecting wood decay caused by at least one kind of wood-destroying fungus selected from the group consisting of Fomitopsis palustris, Gloeophyllum trabeum, Coniophora puteana, Serpula lacrymans, Trametes versicolor, and Gloeophyllum sepiarium.

6. The method for diagnosing wood decay according to 1 or 2, wherein determination through antigen-antibody reaction is carried out by dot-blot method or enzyme-linked immunosorbent assay (ELISA) method.

7. The method for diagnosing wood decay according to 6, using dot-blot method in determination of decay in the wood to be examined, wherein a substrate for dot-blotting, having a porous membrane, is prepared in a device structured to instruct or record spotting positions and the spotting positions of the substrate are spotted with extract of the wood to be examined.

8. The method for diagnosing wood decay according to 7, wherein a substrate for dot-blotting is spotted with a standard sample which has been extracted from wood having a known degree of decay and comparison between the spots of the standard sample and the test sample is conducted to determine the degree of decay of the test sample.

9. An agent used for diagnosing wood decay, which comprises an antibody obtained by sensitizing an animal with an antigen which is a protein having a molecular weight of 1,000 to 100,000 obtained by culturing a wood-destroying fungus and which agent is contacted with extract of wood to be examined in determination of the decay.

10. The agent used for diagnosing wood decay according to 9, wherein the antibody is obtained by sensitizing an animal with a protein having a molecular weight of 1,000 to 100,000 obtained by culturing one wood-destroying fungus selected from the group consisting of Fomitopsis palustris, Gloeophyllum trabeum, Coniophora puteana, Serpula lacrymans, Trametes versicolor and Gloeophyllum sepiarium.

11. The agent used for diagnosing wood decay according to 10, wherein the antibody is obtained by sensitizing an animal with a protein having a molecular weight of 1,000 to 100,000 obtained by culturing Fomitopsis palustris.

12. The agent used for diagnosing wood decay according to any one of 9 to 11, wherein the antibody is obtained from an animal sensitized with an antigen solution containing a protein having a molecular weight of 1,000 to 100,000 obtained by subjecting a culture liquid to ultrafiltration or gel filtration after culturing a wood-destroying fungus in a liquid medium at 10 to 40° C.

13. The agent used for diagnosing wood decay according to any one of 9 to 11, comprising the antibody as antiserum.

14. The agent used for diagnosing wood decay according to any one of 9 to 11, comprising the antibody as purified protein.

15. The agent used for diagnosing wood decay according to 14, comprising the antibody as polyclonal antibody.

16. The agent used for diagnosing wood decay according to any one of 9 to 11, comprising the antibody as monoclonal antibody.

17. A test kit for diagnosing decay in wood, including a substrate for dot-blotting, having a porous membrane prepared in a device structured to instruct or record spotting positions and the diagnosis agent according to any one of 9 to 16.

18. The test kit for diagnosing decay in wood according to 17, wherein the substrate for dot-blotting is spotted with a standard sample extracted from wood having a known decay degree.

(A) Method for Diagnosing Wood Decay

In the method for diagnosing wood decay according to the present invention, wood decay (degree) is determined by evaluating the presence or absence and amounts of metabolic products derived from wood-destroying fungi present in the wood through antigen-antibody reaction conducted by contacting an antibody obtained by sensitizing an animal to a protein as antigen having a molecular weight of 1,000 to 100,000 which has been obtained by culturing a wood-destroying fungus in a liquid medium, with extract obtained from the wood to be examined.

Wood-destroying fungi usable in the present invention include naturally occurring wood-destroying fungi. Preferred examples of wood-destroying fungi include Fomitopsis palustris, Gloeophyllum trabeum, Coniophora puteana, Serpula lacrymans, Trametes versicolor, and Gloeophyllum sepiarium. The most preferred is Fomitopsis palustris.

Culturing is conducted by using a liquid medium. Examples of the culture medium usable therein include various kinds of liquid media, however, particularly preferred is a medium containing cellobiose or containing oligosaccharide or polysaccharide which can produce cellobiose through metabolism of the above fungi, i.e., a medium containing β-glucan as the main carbon source. The cellobiose concentration in the medium is generally within a range of 0.01 to 10 mass %, preferably 0.5 to 5mass %. Generally, in addition to cellobiose, known nitrogen sources (such as ammonium nitrate), thiamine, peptone and if appropriate, yeast extract is added to be contained in the medium and further, trace elements (K, Ca, Mg, B, Mn, Zn, Cu, Mo and Fe) are added.

Any culturing conditions may be employed as far as the above fungi can grow. Generally, shaking culture or stationary culture is performed at 10 to 40° C., preferably at 20 to 30° C., for 5 hours to 6 months, preferably for 1 week to 1 month.

After culturing, proteins of molecular weight range of 1,000 to 1,000,000, preferably 5,000 to 100,000 are fractionated from the culture filtrate. Fractionation can be conducted, for example, by using ultrafiltration membrane or by gel filtration. Any other method may be also employed as far as proteins of the above molecular weight range can be fractionated without being damaged.

An animal is sensitized to thus obtained protein as antigen by conventionally used method.

The protein concentration at the time of sensitization is usually from 0.01 to 100 mg/ml, preferably 0.1 to 10 mg/ml. In preparation of solution containing the antigen, any buffer solution that does not adversely affect living organism may be employed. As a preferred example, PBS (phosphate buffered saline) may be used.

The thus prepared antigen solution may be mixed with an adjuvant at an appropriate mixing ratio, (e.g. 1:1) and used. Preferred examples of adjuvant include those derived from tubercle bacillus, (Freund's complete adjuvant and Freund incomplete adjuvant), RIBI adjuvant, Pertussis adjuvant and inorganic adjuvant (aluminium adjuvant), however, are not limited thereto. Depending on the molecular weight of the protein and the species of the animal to be sensitized, it is not always necessary to use an adjuvant.

Examples of animal to be employed for sensitization include mammals such as rabbit, horse, goat, sheep, guinea pig and mouse, and birds such as duck and chicken. The antigen solution or a mixture of the antigen solution and an adjuvant is injected to the animal subcutaneously or intramuscularly. At intervals of 0.5 to 8 weeks, preferably 1 to 3 weeks, sensitization may be repeated 1 to 6 times. Generally, blood is sampled several weeks after sensitization and the blood sample is subjected to centrifugation to thereby separate blood serum therefrom. For example, the second subcutaneous injection is administered 0.5 to 8 weeks (preferably 1 to 3weeks) after the first sensitization, further, the third subcutaneous injection is administered 0.5 to 8 weeks (preferably 1 to 3 weeks) after the second sensitization, furthermore, the fourth subcutaneous injection is administered 0.5 to 8 weeks (preferably 1 to 3 weeks) after the third sensitization and 0.5 to 4 weeks (preferably 1 to 3 weeks) after the fourth sensitization, whole blood collection is conducted. This immunization schedule is only one example, and immunization may be scheduled discretionarily as far as sufficient immune response against the antigen can be observed.

In the present invention, although thus obtained antiserum may be used as is, the antiserum may be purified through an affinity column such as Protein A, G-Column or the like if necessary. Alternatively, a monoclonal antibody may be prepared by a conventional method.

In the aforementioned document, C. A. Clausen et al. (“Early detection of brown-rot decay in southern yellow pine using immunodiagnostic procedures”, Wood Sci. Technol. 26, 1-8 (1991)), antigens derived from multiple kinds of wood-destroying fungi are mixed and inoculated into a rabbit, to thereby obtain the respective antibodies as a mixture. In the method of the present invention, by using an antibody obtained through inoculation of a protein derived from a single kind of wood-destroying fungus, typical wood-destroying fungi can be detected.

Diagnosis of wood-decay degree is conducted by contacting extract obtained from the test subject sample wood (the wood to be examined) with the above-obtained antibody. Examples of test subject sample wood include a cut piece of wood, a drilled piece of wood, sawdust and wood powder, which are taken out of the wood to be examined. Extraction is conducted by immersing these samples into the following extraction solvent. The extraction solvent is an aqueous solvent preferably prepared by adding to water at least one kind of surfactant such as nonionic surfactant, saccharides, osmotic pressure adjusting agent such as inorganic salts or pH adjuster such as inorganic salts or organic salts. The surfactant is not particularly limited, however, preferred is a surfactant with less protein modification. Examples thereof include Triton-X series (e.g. Triton X-100). Examples of buffer liquid include PBS (phosphate buffered saline), and the pH value is in a range of 4 to 10, preferably from 6 to 9. Extraction time may be determined arbitrarily, and preferred time range is 1 minute to 1 hour. For the purpose of improving extraction efficiency, it is useful to use ultrasonic treatment or homogenizer treatment in combination.

Wood decay is determined through antigen-antibody reaction by using the above antibody solution and the extract from the test subject wood sample. Such a determination is conducted by enzyme-linked immunosorbent assay (ELISA) method, dot-blot method, immunospot method, EIA, immunochromatography or the like, and preferred are ELISA method and dot-blot method.

In the present invention, all methods which can be referred as ELISA method or dot-blot method may be used. Typically, the target antigen (wood-destroying fungi antigen) is searched for by using an antibody solution, and presence or absence of the target antigen in the sample wood is judged by using a marker antibody (antibody for detection). That is, contact is made between the above antibody solution and the extract from the test subject wood sample. In a case where wood-destroying fungi are present in the test subject wood sample, extract therefrom contains the fungi or antigen derived from the fungi and therefore, a complex of the above antibody and the antigen derived from wood-destroying fungi is formed at the contacting part. On the other hand, a labeled antibody is prepared by combining another antibody (e.g., goat anti-rabbit IgG antibody) against the animal (e.g., rabbit) used to obtain the above antibody with an appropriate reporter molecule (enzyme, phosphor, coloring reagent, radioactive isotope or the like) and is reacted with the extract. When the test sample contains the above complex, the complex is further combined with the labeled antibody to thereby form a secondary complex. After removing the labeled antibody which has remained uncombined, the presence or absence and the amount of enzymatic reaction, fluorescence, coloring and radioactivity are determined.

Enzymes (typically peroxidase, alkaline phosphatase, β-galactosidase and the like), coloring agents, radioactive materials and the like used in the present invention are those conventionally used in immunological assays and can be prepared by conventionally known method or those commercially available may be also employed.

Hereinafter, an example of the “ELISA method” procedure is described, however the following assay condition is an only representative range and the assay condition employable in the present invention is not limited to the example.

Onto a microtiter plate made of resin such as polystyrene, polyethylene and polypropyrene, a predetermined amount, for example, 100 μl of extract of the test subject sample wood is added and incubated at 0 to 50° C., preferably 30 to 40° C., for 1 minute to 24 hours, preferably 30 minutes to 3 hours. The microtiter plate is then washed with a buffer liquid (preferably PBS) containing surfactant at arbitrarily selected concentration (preferably 0.05% Tween 20) 1 to 10 times, preferably 2 to 3 times (hereinafter, the procedure is referred to as “washing”.) Next, a blocking buffer liquid (a buffer liquid (preferably PBS) containing a general type of protein, such as BSA (bovine serum albumin)), is added to the microtiter plate and incubated at 0 to 50° C., preferably 30 to 40° C., for 1 minute to 24 hours, preferably 30 minutes to 3 hours. Then, the microtiter plate is subjected to washing.

A predetermined amount, for example, 100 μl of an antibody solution diluted with a buffer liquid (preferably PBS) is added onto a microtiter plate and incubated at 0 to 50° C., preferably 30 to 40° C., for 1 minute to 24 hours, preferably 30 minutes to 3 hours. After washing the microtiter plate, a predetermined amount, for example, 100 μl of a solution of an anti-rabbit IgG antibody of mammals such as rabbit, horse, sheep, guinea pig or mouse, preferably goat, conjugated with an enzyme such as alkaline phosphatase, peroxidase or β-galactosidase is added to the microtiter plate and incubated at 0 to 50° C., preferably 30 to 40° C., for 1 minute to 24 hours, preferably 30 minutes to 3 hours. After washing the microtiter plate, a predetermined amount, for example, 100 μl of a subsrtate (substrate for detection) solution of the enzyme used is added to the microtitler plate and reacted at room temperature for 0.1 minute to 24 hours, preferably for 1 minute to 60 minutes. Absorption is measured by using a spectrophotometer. Any substrate for detection (preferably water-soluble) may be used without particulr limitation as far as the absorption wavelength changes according to reaction with enzyme, and those conventionally employed in ELISA method may be used in the present invention. For example, in a case where alkaline phosphatase is used as enzyme, pNPP (4-nitrophenyl phosphoric acid: the substrate of alkaline phosphatase) can be used.

Dot-blot method, which is a generally employed method, can also be employed and one example is described as follows.

A porous membrane-like material (hereinfter abbreviated as “membrane”) consisting of cellulose or modified cellulose such as nitrocellulose, glass, nylon, polyethylene, polyester, PVDF (polyvinylidene fluoride) or the like is spotted with a predetermined amount, for example, 10 μl, of extract of the test subject wood sample and dried for 0 second to 1 week, preferably for 1 minute to ten minutes. The membrane is washed with a buffer liquid (preferably PBS) containing surfactant (preferably 0.05% Tween20) at an arbitrary concentration, 1 to 10 times, preferably 2 to 3 times. (Hereinafter, the washing procedure is abbreviated as “washing”.)

Subsequently, the membrane is immersed in a blocking buffer liquid (a buffer liquid containing a general type of protein such as BSA (bovine serum alubumin), preferably PBS), and incubated at 0 to 50° C., preferably 30 to 40° C., for 1 minute to 24 hours, preferably 30 minutes to 3 hours. The membrane is subject to washing. The membrane is immersed in an antibody solution diluted with a buffer liquid,preferably PBS, and incubated at 0 to 50° C., preferably 30 to 40° C., for 1 minute to 24 hours, preferably 30 minutes to 3 hours. The membrane is subject to washing. The membrane is immersed in a solution of an anti-rabbit IgG antibody of mammals such as rabbit, horse, sheep, guinea pig or mouse, preferably goat, conjugated with an enzyme such as alkaline phosphatase, peroxidase or β-galactosidase and incubated at 0 to 50° C., preferably 30 to 40° C., for 1 minute to 24 hours, preferably 30 minutes to 3 hours. The membrane is subject to washing. The membrane is immersed in a subsrtate (substrate for detection) solution of the enzyme used and reacted at room temperature for 0.1 minute to 24 hours, preferably for 1 minute to 60 minutes. Color changing is observed at the spotted positions. The substrate for detection used is not particularly limited as far as absorption wavelength changes and water-solubility decreases according to reaction with the enzyme, and those conventionally employed in dot-blot method may be employed. For example, in a case where alkaline phosphatase is used as enzyme, BCIP/NBT (chromogenic premixed substrate for alkaline phosphatase) may be used.

(B) Wood-Decay Diagnostic Agent

Thus, the antiserum, purified protein or polyclonal antibody obtained by sensitizing an animal with a protein as antigen having a molecular weight of 1,000 to 100,000, preferably 5,000 to 100,000 obtained by liquid-culturing naturally occuring wood-destroying fungi can be conjugated with an appropriate detection antibody against the sensitized (immune) animal for detection and used as agent for diagnosing wood decay.

Also, in a case of polyclonal antibody or monoclonal antibody, by immobilizing the antibody on a styrene bead or the like, a product used in diagnosis of wood decay which is determined by presence or absence of aggregate of the antibody can be prepared.

(C) Simple Diagnosis Method of Wood Decay and Test Kit Used in the Simple Diagnosis Method

Diagnosis of wood decay according to the present invention can be conducted more simply and precisely by combination with the following substrate for diagnosis.

That is, a plate made of a resin such as polystyrene, polyethyrene or polypropylene on which a groove of 0.5 to 10 cm in width, 1 to 50 cm in length and 0.1 to 10 cm in depth, preferably 1 to 5 cm in width, 5 to 20 cm in length and 0.5 to 2 cm in depth is provide is prepared. The form of the groove may be either a bottomed form (like a container) or a bottomless form (hereinafter the plate is referred to as “device”). The membrane-like material (hereinafter abbreviated as “membrane”) of cellulose or modified cellulose such as nitrocellulose, glass, nylon, polyethylene, polyester or PVDF is cut in a form to fit in the groove of the device, and placed in the groove. By allowing at least a part of the membrane except for positions to be spotted to be in contact with the inner surface of the groove in this step, the operations thereafter can be smoothly conducted.

In diagnosis of decay degree of wood, test is conducted with the above device as basal plate. That is, according to the aforementioned method for diagnosing wood decay, test subject samples are taken from several points (e.g. 5 points) where decay is suspected in wood in a wooden building such as a house and extracts are obtained from the respective samples. The membrane placed in the groove is spotted with the respective extracts of the five test samples, and the determination of decay is made as follows according to the aforementioned dot-blot method.

In this case, spotting is conducted by dropping the extract on the membrane with appropriate intervals between spots and for easy observation of color changes in spotted portions, the form of the groove in the device may be designed according to the spotted positions. For example, scale lines or diamond marks may be provided on the plate surface between the grooves in the device, so that spotting can be accelerated with optimum intervals according to the predetermined number of spots, and spaces for writing down the respective sample numbers to identify each spot. By doing so, spotted positions in the membrane and correspondence of one spot with one sample can be clearly indicated, whereby multiple samples can be precisely examined at the same time by using a single device. Moreover, it is preferable to prepare a standard spot on the plate or membrane by using a sample wood whose decay degree is known. That is, presence or absence of decay caused by wood-destroying fungi can be judged from the color change of each spot and the decay degree can be quantitatively evaluated by comparison in absorption (or comparison in contrasting density when visually observed by naked eye) between the spots of test subject sample woods and the standard spot thus prepared in advance. Such a standard spot can be provided by spotting a standard sample obtained from a wood whose decay degree is already known by decrease in weight or the like (e.g. wood of weight loss rate of 1%, 3%, 5%, 7% or 10%). A single standard spot may be provided or multiple standard spots with different decay degrees may be provided.

Further, the device may be a combination of a basal plate on which a groove is provided as above and a cover plate with an opening having a shape of circle, oval or rectangle. The width of the cover plate is almost the same with the width of the groove so that the cover plate fit with the groove. In conducting the spotting step, the membrane is covered by the cover plate and the spotting of the extract is conducted within the opening of the cover plate. In washing step, the cover plate is taken off and in the final determination step, the cover plate is used again to cover the membrane. In this case, by marking the sample numbers at the opening portions or providing spaces for writing down correspondence of one spotted position with one sample, each spot can be clearly identified as a certain sample.

Method of diagnosing wood decay and diagnosing agent according to the present invention is effective on various types of wood. Examples of wood to which the Method of diagnosing wood decay and diagnosing agent according to the present invention is applicable include pines such as red pine and larch, cedar, hemlock and cypress. Moreover, examples of wood-destroying fungi detectable by the present invention include Fomitopsis palustris, Gloeophyllum trabeum, Coniophora puteana, Serpula lacrymans, Trametes versicolor and Gloeophyllum sepiarium.

According to the present invention, presence or absence of wood decay caused by wood-destroying fungi can be easily determined in a short period of time. In the method of the present invention, which can determine wood decay specifically caused by wood-destroying fungi and does not include response to fungi present on the wood surface and metabolites therefrom, possibility of wrong diagnosis is extremely low. Furthermore, the agent for detecting wood-destroying fungi, which is obtained by sensitizing an animal with a single species of wood-destroying fungus, can be easily prepared and therefore production of the agent is easy and uniform products can be supplied easily.

### BRIEF DESCRIPTION OF THE DRAWINGS

FIG. 1 is a graph showing changes in absorption by species of the fungus used in Example 2.

### BEST MODE FOR CARRYING OUT THE INVENTION

Hereinafter, the present invention will be described by Examples and Comparative Examples. However, the present invention should by no means be limited to these examples.

### EXAMPLE 1

### Preparation of Antibody

(1) Preparation of Antigen

50 ml of liquid medium (pH 5.5) which contains 1% cellobiose, 0.2% NH₄NO₃, 0.2% KH₂PO₄, 0.05% MgSO₄.7H₂O, 0.01% CaCl₂.2H₂O, 0.57 ppm H₃BO₄, 0.036 ppm MnCl₂.4H₂O, 0.31 ppm ZnSO₄.7H₂O, 0.039 ppm CuSO₄.5H₂O, 0.018 ppm (NH₄)₆Mo₇O₂₄.4H₂O, 0.015 ppm FeSO₄.7H₂O, 1 ppm thiamine hydrochloride, 0.5% peptone and 0.05% yeast extract was added into a 500 ml flask for culture, the flask was plugged with cotton and sterilized in an autoclave at 121° C. for 30 minutes.

Fomitopsis palustris (Berk. Et Curt.) Gilbn. & Ryv. FFPRI 0507 was inoculated in this medium, statically cultured at 27° C. for 2 weeks and the obtained cultured solution was filtered by a glass filter, to thereby obtain cultured filtrate. The cultured filtrate was filtered by an ultrafilter membrane (Ultrafree-15, Biomax 100, a membrane-attached unit by Millipore Corporation), to thereby obtain a fraction having a molecular weight of 100,000 or less. This fraction was concentrated by a ultrafilter membrane (Ultrafree-15, Biomax 5, a membrane-attached unit, by Millipore Corporation) and washed with PBS of the equivalent amount 3 times, to thereby obtain a faction having a molecular weight of 5,000 to 100,000. The obtained fraction was diluted with PBS to thereby have a protein concentration of about 0.7 mg/ml, and the resultant was used as antigen solution.

(2) Preparation of Polyclonal Antibody

Polyclonal antibody was prepared by using the antigen solution obtained in (1). A Freund complete adjuvant was mixed with the antigen solution at a ratio of 1:1. This solution was subcutaneously injected to a rabbit in 10 points. After 2 weeks, subcutaneous injection was administered again. Further, after another 2 weeks, subcutaneous injection was administered again. After another week, whole blood was collected. Whole blood was left to stand for 30 minutes, and the antiserum was separated through centrifugation. The obtained antiserum was purified by Protein A affinity column (by Protein A Kit, by SIGMA-ALDRICH), to thereby obtain a polyclonal antibody solution.

### EXAMPLE 2

### Diagnosis by Using ELISA Method

(1) Preparation of Decayed Wood Chips and Extract from the Decayed Wood Chips

As culture substrates, 10 pieces of cedar splint wood and 20 ml of pure water were placed in a 500 ml-volume Erlenmeyer flask, and after plugged with cotton, the medium was sterilized in an autoclave at 121° C. for 30 minutes.

In this way, 7 of such an Erlenmeyer flask were prepared and strains of 7 kinds of fungi, Fomitopsis palustris, Gloeophyllum trabeum, Coniophora puteana, Serpula lacrymans, Trametes versicolor, which are wood-destroying fungi, Penicillium funiculosum (which is used in JIS fungus resistance test) which is a wood surface contaminant fungus and Gliocladium virens Miller (which is used in JIS fungus resistance test, former name: Trichoderma viride) were respectively inoculated, and kept at 24° C. for 4 weeks.

After 4 weeks, from thus decayed wood pieces, small chip-like wood samples were taken our by using a manual drill. To the samples, 5 ml of PBS (pH 7.4) which contained 0.1% Triton X-100 per 1 g of the wood sample was added, and the resultant was left to stand for 10 minutes, to thereby obtain extract from the test subject wood chip samples.

(2) Determination of Wood Decay by ELISA Method

Onto a microtiter plate made of polystyrene (NUNC-IMMUNO PLATE, by Nalge Nunc International K.K.), 100 μl of the extract from each of the test subject wood samples and a control (extract only) were added.

The plate was incubated at 37° C. for 1 hour. Subsequently, the microtiter plate was washed twice with PBS which contained 0.05% Tween 20 (hereinafter, this operation is referred to as “washing”). Next, 100 μl of blocking buffer liquid (PBS which contained 1% BSA and 0.05% Tween 20) was added to the microtiter plate and incubated at 37° C. for 1 hour. Then, the microtiter plate was subject to washing.

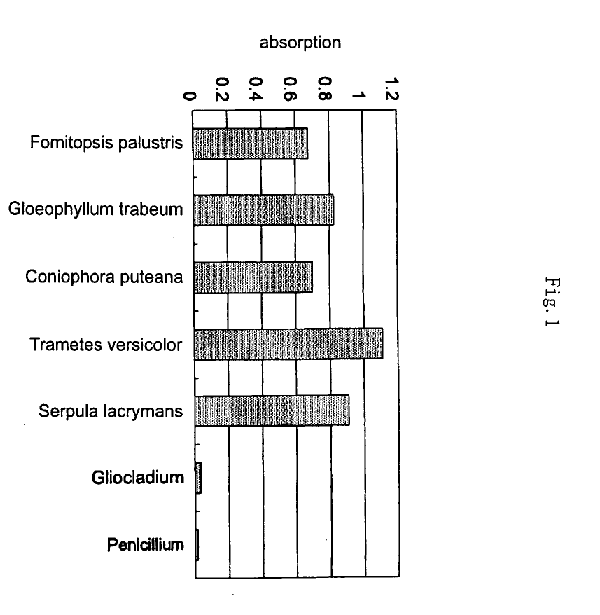

The antibody solution obtained in (2) of Example 1 was diluted with PBS, and then 100 μl of the solution was added to the microtiter plate, and the plate was incubated for 1 hour. Subsequently, the microtiter plate was subject to washing. After 100 μl of alkaline phosphatase-conjugated goat anti-rabbit IgG antibody solution (by SIGMA) was added to the microtiter plate, the microtiter plate was incubated at 37° C. for 1 hour. Then the microtiter plate was subject to washing. 100 μl of 0.1% pNPP (4-nitrophenyl phosphate) solution was added to the microtiter plate, to thereby cause reaction at room temperature for 30 minutes. The absorption at 405 nm was measured by a spectrophotometer, and FIG. 1 shows the results.

As a result, a significant change in absorption was observed with respect to Fomitopsis palustris, Gloeophyllum trabeum, Coniophora puteana, Serpula lacrymans and Trametes versicolor, while no significant change in absorption was found with respect to Penicillium funiculosum and Gliocladium virens Miller, as compared with the control.

By ELISA method which used the antibody proposed in the present invention, it was revealed that wood material decayed by 5 typical kinds of wood destroying fungi can be detected by the method of the invention and that the method is a method which is specific to detection of wood decay caused by wood destroying fungi without detecting other fungi such as wood surface contaminant fungi.

### EXAMPLE 3

### Determination of Wood Decay by Dot-Blot Method

The wood extract was prepared in the same manner as in Example 2, and 10 μl of the extract from each of the test subject wood samples and a control were spotted to a nitrocellulose membrane (0.451 μl of nitrocellulose membrane, product of BIO-RAD Laboratories, Inc., hereinafter simply abbreviated to as “membrane”). This membrane was dried for 10 minutes. The membrane was washed twice with PBS which contained 0.05% Tween 20 (hereinafter, this operation is referred to as “washing”). Then, the membrane was immersed in a blocking buffer liquid (PBS which contained 1% BSA and 0.05% Tween 20), incubated at 37° C. for 10 minutes and the membrane was subjected to washing.

The antibody solution obtained in (2) of Example 1 was diluted with PBS, the membrane was immersed in the solution, and the membrane was incubated at 37° C. for 10 minutes. After washing the membrane, the membrane was immersed in alkaline phosphatase-conjugated goat anti-rabbit IgG antibody solution (manufactured by SIGMA). Then, the membrane was incubated at 37° C. for 10 minutes. After washing the membrane, the membrane was immersed in a BCIP/NBT solution (an AP coloring kit, by BIO-RAD Laboratories, Inc.) to thereby cause reaction for 10 minutes at room temperature. Color change on the spotted positions was observed. The results are shown in Table 1.

TABLE 1

| type of fungus | color change |
|---|---|
| Fomitopsis palustris | ⊚ |
| Gloeophyllum trabeum | ⊚ |
| Coniophora puteana | ⊚ |
| Trametes versicolor | ⊚ |
| Serpula lacrymans | ⊚ |
| Gliocladium | x |
| Penicillium | x |

⊚: remarkable color change
x: no significant color change

By dot-blot method which used the antibody proposed in the present invention, it was revealed that wood material decayed by 5 typical kinds of wood destroying fungi can be detected by the method of the invention and that the method is a method which is specific to detection of wood decay caused by wood destroying fungi without detecting other fungi such as wood surface contaminant fungi (Penicillium funiculosum and Gliocladium virens). Further, it was revealed that decay of the wood to be examined can be determined more quickly by employing dot blot method. Furthermore, by using a diagnosis kit/method where a standard sample spot is provided, the degree of wood decay can be easily evaluated quantitatively with high reliability.

### INDUSTRIAL APPLICABILITY

The agent for diagnosing wood decay of the present invention, which shows a clear response not only to wood decay caused by brown-rot fingi such as Fomitopsis palustris but also to wood decay caused by white-rot fungi such as Trametes versicolor, is useful for determination of decay degrees in building materials and stored wood materials. Further, The agent for diagnosing wood decay of the present invention, which is obtained by sensitizing an animal to a single kind of a wood-destroying fungus, can be not only produced easily but also supplied as uniform product, so that the agent can be widely used in general building materials.

1. A method for diagnosing wood decay, wherein wood decay is judged through antigen-antibody reaction of contacting extract of wood to be examined with an antibody obtained by sensitizing an animal with an antigen which is a protein having a molecular weight of 1,000 to 100,000 obtained by culturing a wood-destroying fumgus.

2. The method for diagnosing wood decay according to claim 1, wherein decay by multiple kinds of wood-destroying fungi is judged through antigen-antibody reaction of contacting extract of wood to be examined with an antibody obtained by sensitizing an animal with an antigen which is a protein having a molecular weight of 1,000 to 100,000 obtained by liquid-culturing a wood-destroying fungus.

3. The method for diagnosing wood decay according to claim 1, wherein the protein is obtained by culturing one wood-destroying fungus selected from the group consisting of Fomitopsis palustris, Gloeophyllum trabeum, Coniophora puteana, Serpula lacrymans, Trametes versicolor and Gloeophyllum sepiarium.

4. The method for diagnosing wood decay according to claim 3, wherein the protein is obtained by culturing Fomitopsis palustris.

5. The method for diagnosing wood decay according to claim 1, detecting wood decay caused by at least one kind of wood-destroying fungus selected from the group consisting of Fomitopsis palustris, Gloeophyllum trabeum, Coniophora puteana, Serpula lacrymans, Trametes versicolor, and Gloeophyllum sepiarium.

6. The method for diagnosing wood decay according to claim 1, wherein determination through antigen-antibody reaction is carried out by dot-blot method or enzyme-linked immunosorbent assay (ELISA) method.

7. The method for diagnosing wood decay according to claim 6, using dot-blot method in determination of decay in the wood to be examined, wherein a substrate for dot-blotting, having a porous membrane, is prepared in a device structured to instruct or record spotting positions and the spotting positions of the substrate are spotted with extract of the wood to be examined.

8. The method for diagnosing wood decay according to claim 7, wherein a substrate for dot-blotting is spotted with a standard sample which has been extracted from wood having a known degree of decay and comparison between the spots of the standard sample and the test sample is conducted to determine the degree of decay of the test sample.

9. An agent used for diagnosing wood decay, which comprises an antibody obtained by sensitizing an animal with an antigen which is a protein having a molecular weight of 1,000 to 100,000 obtained by culturing a wood-destroying fumgus and which agent is contacted with extract of wood to be examined in determination of the decay.

10. The agent used for diagnosing wood decay according to claim 9, wherein the antibody is obtained by sensitizing an animal with a protein having a molecular weight of 1,000 to 100,000 obtained by culturing one wood-destroying fungus selected from the group consisting of Fomitopsis palustris, Gloeophyllum trabeum, Coniophora puteana, Serpula lacrymans, Trametes versicolor and Gloeophyllum sepiarium.

11. The agent used for diagnosing wood decay according to claim 10, wherein the antibody is obtained by sensitizing an animal with a protein having a molecular weight of 1,000 to 100,000 obtained by culturing Fomitopsis palustris.

12. The agent used for diagnosing wood decay according to claim 9, wherein the antibody is obtained from an animal sensitized with an antigen solution containing a protein having a molecular weight of 1,000 to 100,000 obtained by subjecting a culture liquid to ultrafiltration or gel filtration after culturing a wood-destroying fungus in a liquid medium at 10 to 40° C.

13. The agent used for diagnosing wood decay according to claim 9, comprising the antibody as antiserum.

14. The agent used for diagnosing wood decay according to claim 9, comprising the antibody as purified protein.

15. The agent used for diagnosing wood decay according to claim 14, comprising the antibody as polyclonal antibody.

16. The agent used for diagnosing wood decay according to claim 9, comprising the antibody as monoclonal antibody.

17. A test kit for diagnosing decay in wood, including a substrate for dot-blotting, having a porous membrane prepared in a device structured to instruct or record spotting positions and the diagnosis agent according to claim 9.

18. The test kit for diagnosing decay in wood according to claim 17, wherein the substrate for dot-blotting is spotted with a standard sample extracted from wood having a known decay degree.
